(19) United States
(12) Patent Application Publication
(10) Pub. No.: US 2011/0130731 A1
(43) Pub. Date: Jun. 2, 2011

(54) FLUID ARRESTING COVER DEVICES INCORPORATING A WEAK BASE

(76) Inventor: Paul Michael Gottschalk, San Antonio, TX (US)

(21) Appl. No.: 12/627,849

(22) Filed: Nov. 30, 2009

(51) Int. Cl. A61F 13/15; A61L 2/232

(52) U.S. Cl. 604/359; 422/5

(57) ABSTRACT

A diaper or other fluid arresting cover for a human body, bearing a weak base such as sodium bicarbonate for neutralizing acids in fluids secreted by the human body and intercepted by the fluid arresting cover. The fluid arresting cover may comprise in respective order a first water permeable wicking stratum, a second water permeable wicking stratum of different constituency than the first wicking stratum, a third stratum for absorbing water, a fourth stratum which is water permeable and can wick water to other strata, and a liquid impermeable outer membrane. The weak base may be incorporated into the third stratum or alternatively into the third and other strata. The weak base may be impregnated throughout a stratum or may comprise an exterior coating with adhesive engaging the exterior coating.

### FIELD OF THE INVENTION

The present invention relates to diapers and like devices for intercepting fluid discharges from the body, and more particularly, to diapers and like devices which incorporate weak bases for neutralizing acidic bodily discharges.

### BACKGROUND OF THE INVENTION

Diaper rash is a common affliction which affects babies. While diaper rash may have any of a number of causes, one cause is irritation of the skin due to the mildly acidic nature of urine retained by a diaper. Urine can vary in pH so as to be acidic, basic, or neutral, but when acidic, can cause diaper rash. However, for persons other than vegetarians, the pH of urine tends to acidic. Moreover, sleep causes decreased pulmonary ventilation, which results in respiratory acidosis. Therefore, urine excreted during sleep, or upon waking, is usually highly acidic.

Compounds such as bicarbonate of soda have been incorporated into diapers, for example to overcome odors. Diaper constructions incorporating bicarbonate of soda or other weak bases as seen in the present invention are not known.

### SUMMARY OF THE INVENTION

The present invention provides a construction for a fluid arresting cover device which advantageously incorporates a weak base in a way so as to chemically react with and neutralize acids. The weak base may also absorb or otherwise suppress odors. The weak base may comprise for example sodium bicarbonate, sodium carbonate, or any other similarly acting weak base incorporated into one or more layers or strata of the fluid arresting cover device, or coupled to the surface of one or more layers or strata.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is an exaggerated, exploded cross sectional view of a fluid arresting cover device such as a diaper, according to at least one aspect of the invention.

FIG. 2 is an exaggerated perspective view of one component which may be utilized in a fluid arresting cover device such as a diaper, according to at least one further aspect of the invention.

FIG. 3 is an exaggerated side cross sectional view of another component which may be utilized in a fluid arresting cover device such as a diaper, according to at least one further aspect of the invention.

FIG. 4 is a block diagram of steps of a method of providing odor control to a fluid arresting cover device such as the fluid arresting cover device of FIG. 1.

FIG. 5 is a block diagram of steps of a method of neutralizing acidic water in a water bearing bodily discharge, as is performed by the fluid arresting cover device of FIG. 1.

### DETAILED DESCRIPTION

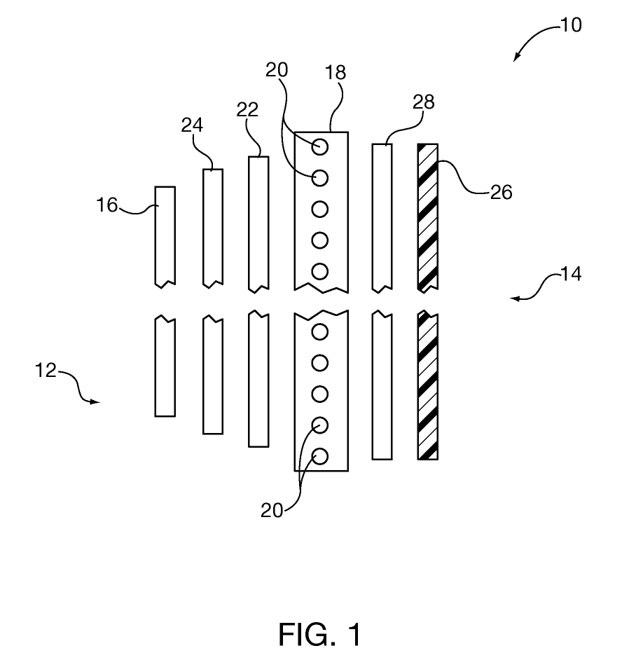

Referring first to FIG. 1, according to at least one aspect of the invention, there is shown one exemplary construction of a fluid arresting cover device 10 for containing bodily discharges containing water from a human body (not shown). A fluid arresting cover device such as the fluid arresting cover device 10 is an article intended to be placed against and cover portions of the human body for the purpose of intercepting and containing fluid discharges. Diapers for babies provide one but not the exclusive example of such fluid arresting cover devices. Bandages, incontinence pads, products for accommodating menses, and still other purposes may be addressed by a fluid arresting cover device according to one or more aspects of the invention.

The fluid arresting cover device 10 may be said to have an interior surface 12 disposed to face the body when the fluid arresting cover device 10 is placed against the human body, and a corresponding exterior surface 14 disposed to face away from the body when the fluid arresting cover device 10 is placed against the human body. Designation of interior and exterior are for semantic purposes in explaining the invention, and will be understood to refer to orientation in facing towards or away from the body of the user when the fluid arresting cover device 10 is placed in operative position thereagainst. The terms interior and exterior may be presented herein alone or used in combination with terms such as surface or side to similar effect. Illustratively, the interior, an interior side, and an interior surface may be utilized interchangeably herein.

Because of the purpose of the invention, elements of the fluid arresting cover device 10 located towards the interior surface 12 are fluid transmissive. The element of the fluid arresting cover device 10 located at the exterior surface 14 is fluid impermeable, so that the bodily discharges are contained and not permitted to foul the environment outside the body.

The fluid arresting cover device 10 may comprise the following flexible strata. A flexible first stratum 16 located at or defining the interior surface 12 may comprise a first water transfer substance (not separately shown) disposed to transport a water based liquid by wicking action. The first water transfer substance, which may be any conventional material known for use with diapers for example, may comprise a solid having interstitial spaces and which does not change form when absorbing and transferring water. For example, the first water transfer substances may comprise a woven or non-woven web of natural or artificial fibers, or a combination of these characteristics, wherein the interstitial spaces are those voids existing between adjacent fibers.

A flexible second stratum 18 may be disposed between the flexible first stratum 16 and the exterior surface 14. The flexible second stratum 18 may comprise a first water absorbent substance which ultimately becomes a gel when absorbing water, wherein the first water absorbent substance may be any known substance or construction conventionally used in diapers. A distinction may be drawn between the first water transfer substance and the first water absorbent substance despite the fact that both may be said to transfer water. The distinction is that the first water transfer substance retains its essential physical configuration when wetted, for example, being a fibrous mass, despite filling or partial filling of interstitial spaces when water is encountered, whereas the first water absorbent substance substantially changes from a generally solid substance to a generally fluid, amorphous substances when water is encountered.

The second stratum 18 may be impregnated with a weak base disposed to react chemically with acidic water so as to neutralize the acidic water, within limits of the nature and quantity of the weak base and the acidic strength and quantity of acidic water present in the bodily discharge. For example, granules 20 of a weak base such as sodium bicarbonate may be introduced into the second stratum 18 at the time of fabrication of the second stratum 18. A weak base is a substance that when combined with water may develop a pH value between seven and about nine, and may comprise for example the aforementioned sodium bicarbonate, sodium carbonate, or any other similarly acting weak base. The granules 20 are shown in representative fashion in FIG. 1, and are therefore to be understood not to be to scale. In a similar vein, all components of the fluid arresting cover device 10 are shown in exaggerated or representative fashion, and are not necessarily drawn to scale.

A flexible third stratum 22 may be disposed beside the flexible second stratum 18 and may be separated from the flexible first stratum 16 by the flexible second stratum 18. The flexible third stratum 22 may comprise a second water transfer substance which comprises a solid having water retaining interstitial spaces and which does not change form when absorbing water, and may for example be of similar or identical constituency as that of the flexible first stratum 16.

The fluid arresting cover device 10 may comprise a flexible fourth stratum 26 located on or at the exterior side 14 of the fluid arresting cover device 10. The flexible fourth stratum 26 may comprise for example a thin plastic or polymeric sheet. The flexible fourth stratum 26 functions as a water impermeable barrier for preventing escape to the outside of a water containing fluid by the fluid arresting cover device 10.

The fluid arresting cover device 10 may comprise a flexible fifth stratum 24 disposed between the flexible first stratum 16 and the flexible second stratum 22. The flexible fifth stratum 24 may comprise a third water transfer substance disposed to transport a water based liquid by wicking action. The third water transfer substance may be different in constituency, density, or any other characteristic from the first water transfer substance and the second water transfer substance. This arrangement of three strata and their water transfer characteristics may be similar or identical to corresponding arrangements which are known in diapers, and therefore need not be further detailed herein. It must be stressed that an important difference between the present invention and conventional known diapers lies in the use of the weak base rather than in utilization of any particular arrangement of strata or plies in forming the fluid arresting cover device 10. Material may be selected for one or more strata, for example, for its directional or other permeability characteristics relative to inhibition of particle migration.

The fluid arresting cover device 10 may comprise a flexible sixth stratum 28 disposed between the flexible second stratum 18 and the flexible fourth stratum 26. The flexible sixth stratum 28 may be of construction similar to that of the flexible first stratum 16 or the flexible stratum 22 for example. The flexible sixth stratum 28 may assist in maintaining consistent thickness of the fluid arresting cover device 10 after the flexible second stratum 18 becomes a gel, or may increase maximum fluid retention capacity, for example.

It will be noticed that the various flexible strata such as the flexible first stratum 16, the flexible third stratum 22, the flexible fifth stratum 24, and the flexible sixth stratum 28 have been presented out of the order shown in FIG. 1. This is because while the fluid arresting cover device 10 may comprise these elements, another fluid arresting cover device (not shown) according to other possible aspects of the invention may omit some of these elements, and may for example comprise a construction equivalent to including only the first flexible stratum 16, the flexible second stratum 18, and the fourth flexible stratum 26. For example, the arrangement including the flexible first stratum 16, the flexible third stratum 22, and the flexible fifth stratum 24 may be regarded as advantageous in wicking a water based fluid to the flexible second stratum 18 more rapidly than for example a single stratum (not shown) of similar dimensions but of homogeneous constituency. The invention could nevertheless be practiced with different arrangements of wicking strata, such as that utilizing the single stratum. Hence in some constructions of a fluid arresting cover device according to the invention, there could be only first, second, and third strata such as the flexible first stratum 16, the flexible second stratum 18, and the flexible third stratum 26. For semantic reasons, it is preferred to order the various strata such that there will always be first, second, and third strata regardless of presence of other strata.

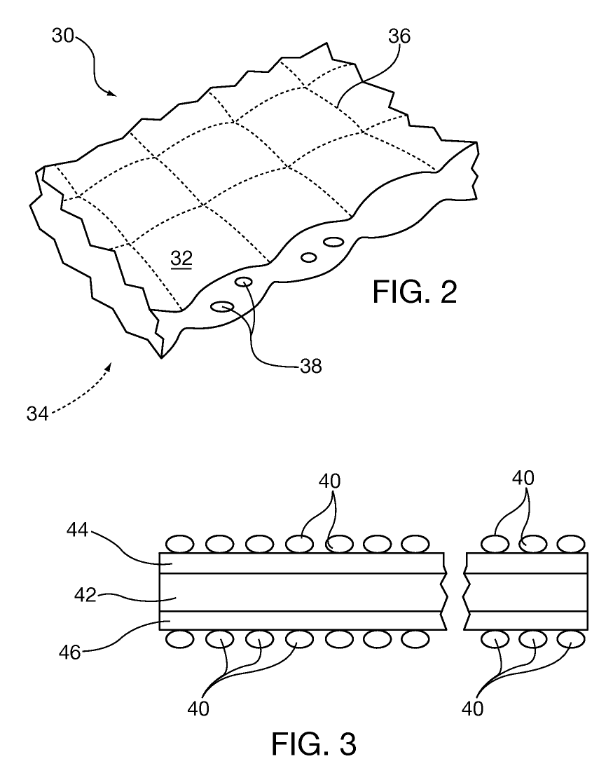

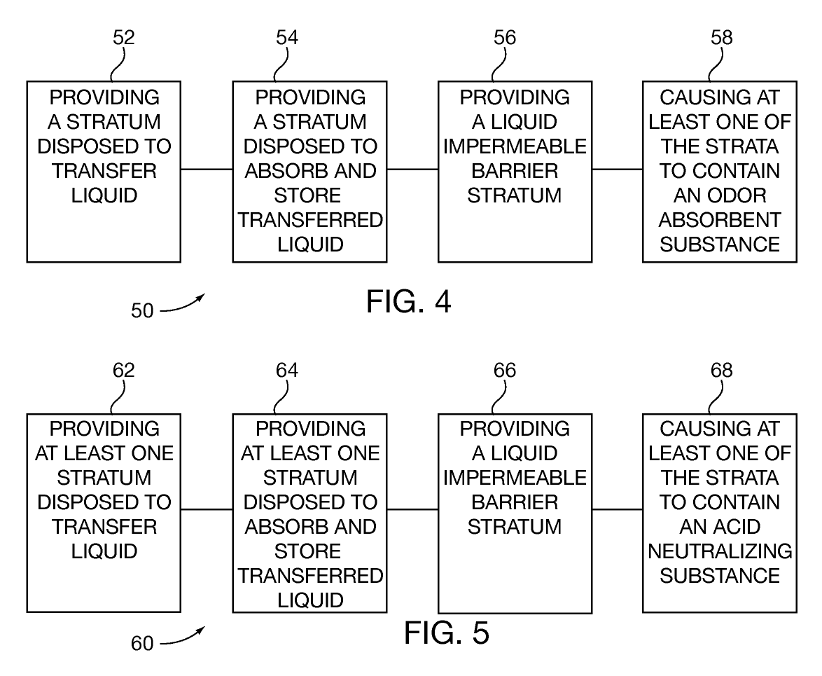

FIG. 2 shows a possible construction for forming the flexible first stratum 16, the flexible third stratum 22, the flexible fifth stratum 24, the flexible sixth stratum 28, or of course, any combination of these. A flexible stratum 30, which will be understood to represent any of the flexible first stratum 16, the flexible third stratum 22, the flexible fifth stratum 24, and the flexible sixth stratum 28, may comprise a first web 32, an opposed second web 34 disposed generally parallel to the first web and secured thereto by quilting 36, and a weak base 38 in granular form entrapped between the first web 32 and the second web 34. The first web 32 and the second web 34 collectively form end barriers for securing a suitable substance such as a fibrous woven or non-woven mass in place for the purpose of wicking a water based fluid to the flexible second stratum 18, and for holding the woven or non-woven mass in generally constant thickness despite body movements and other potential disruptions to the fluid arresting cover device 10.

Quilting 36 is a term introduced herein for describing a way of affixing the first web 32 to the second web 34 wherein a fastener is disposed along intersecting lines so as to contact the first web 32 and the second web 34 at spaced apart intervals such as according to the pattern depicted in FIG. 2. The fastener may comprise a filament which is mechanically stitched, or a bead generated by sonic or ultrasonic welding the first web 32 to the second web 34, chemical bonding of the first web 32 and the second web 34, application of an adhesive, or in other ways (none shown). The visual effect of such spacing and arrangement of intersecting lines of the fastener is similar to that used in blankets and the like of the type popularly known as quilts. Hence a construction utilizing the fastener arrangement described above will be referred to as quilting. In quilted material, typically, the constituent material billows out or increases in depth or thickness (i.e., the distance between the first web 32 and the second web 34) where there is no fastener, and is of minimal depth or thickness at the location of the fastener.

FIG. 3 shows a further way in which granules of a weak base 40 in granular form may be coupled to a stratum 42. The stratum 42, which in other ways may be similar to for example the flexible first stratum 16, the flexible third stratum 22, the flexible fifth stratum 24, or the flexible sixth stratum 28, may comprise in addition a first outer coating 44 of adhesive and a second outer coating 46 of adhesive. The coatings 44, 46 of adhesive may be discontinuous along the surface of the stratum 42, for example being perforated or otherwise partially open so as to be liquid permeable and able to pass fluids to the interior of the stratum 42. Granules of the weak base 40 may be adhered to the coatings 44 and 46 such that the weak base 40 is disposed on the outer surface of the stratum 42. The various strata of a fluid arresting cover device such as the fluid arresting cover device 10 may utilize the constructions seen in any of FIGS. 1, 2, and 3, and in any combination thereof. Therefore, even if the flexible second stratum 18 is internally impregnated with the weak base 20, in other strata, at least some of the weak base may be disposed on the outer surface of the other strata.

The invention may be thought of as a method 50 of providing odor control to a fluid arresting cover device such as the fluid arresting cover device 10. The method 50 may comprise a step 52 of providing at least one stratum disposed to transfer liquid, a step 54 of providing at least one stratum disposed to absorb and store transferred liquid, a step 56 of providing a liquid impermeable barrier stratum, and a step 58 of causing at least one of the strata to contain an odor absorbent substance. The odor absorbent substance may be for example sodium bicarbonate as seen with the granules 20 of the weak base, or the weak base 38 or 40.

The invention may further be thought of as a method 60 of neutralizing acidic water in a water bearing bodily discharge, comprising a step 62 of providing at least one stratum disposed to transfer liquid, a step 64 of providing at least one stratum disposed to absorb and store transferred liquid, a step 66 of providing a liquid impermeable barrier stratum, and a step 68 of causing at least one of the strata to contain an acid neutralizing substance. The acid neutralizing substance may be for example sodium bicarbonate as seen with the granules 20 of the weak base, or the weak base 38 or 40.

The apparatuses mentioned in the steps of either the method 50 or the method 60 may comprise the apparatuses of FIGS. 1, 2, and 3.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A fluid arresting cover device for containing bodily discharges containing water from a human body, the fluid arresting device having an interior surface disposed to face the body when the fluid arresting cover device is placed against the human body and an exterior surface disposed to face away from the body when the fluid arresting cover device is placed against the human body, comprising:
a flexible first stratum comprising a first water transfer substance disposed to transport a water based liquid by wicking action, which comprises a solid having water retaining interstitial spaces and which does not change form when absorbing and transferring water;
a flexible second stratum disposed between the flexible first stratum and the exterior surface of the fluid arresting cover device, comprising a first water absorbent substance which ultimately becomes a gel when absorbing water, wherein the second stratum is impregnated with a weak base disposed to react chemically with acidic water so as to neutralize the acidic water; and
a flexible fourth stratum comprising a water impermeable barrier which is located on the exterior side of the fluid arresting cover device.

2. The fluid arresting cover device of claim 1, further comprising a flexible third stratum disposed beside the flexible second stratum and separated from the flexible first stratum by the flexible second stratum, comprising a second water transfer substance which comprises a solid having water retaining interstitial spaces and which does not change form when absorbing water.

3. The fluid arresting cover device of claim 1, wherein the first water transfer substance of said flexible first stratum comprises substantially the same constituent material of the second water transfer substance of the flexible third stratum.

4. The fluid arresting cover device of claim 3, further comprising a flexible fifth stratum disposed between the flexible first stratum and the flexible second stratum, wherein the flexible fifth stratum comprises a third water transfer substance disposed to transport a water based liquid by wicking action, wherein
the first water transfer substance is substantially identical in constituency to the second water transfer substance, and
the third water transfer substance is different from the first substance and the second substance.

5. The fluid arresting cover device of claim 1, wherein at least some of the weak base is disposed on the outer surface of the flexible second stratum.

6. The fluid arresting cover device of claim 5, wherein the flexible second stratum is coated with a liquid permeable adhesive, and at least some of the weak base is engaged by the liquid permeable adhesive.

7. The fluid arresting cover device of claim 1, wherein the flexible second stratum comprises a first web, a second web disposed generally parallel to the first web and secured thereto by quilting, and at least some of the weak base is in granular form and is entrapped between the first web and the second web.

8. The fluid arresting cover device of claim 1, wherein at least some of the weak base is disposed upon the flexible first stratum.

9. The fluid arresting cover device of claim 8, wherein the flexible first stratum is coated with a liquid permeable adhesive, and at least some of the weak base is engaged by the liquid permeable adhesive coated onto the flexible first stratum.

10. The fluid arresting cover device of claim 1, wherein at least some of the weak base is disposed upon the flexible third stratum.

11. The fluid arresting cover device of claim 10, wherein the flexible third stratum is coated with a liquid permeable adhesive, and at least some of the weak base is engaged by the liquid permeable adhesive coated onto the flexible third stratum.

12. The fluid arresting cover device of claim 1, wherein the weak base is selected from the group consisting of sodium bicarbonate and sodium carbonate.

13. A method of providing odor control to a fluid arresting cover device such as a diaper, comprising the steps of:
providing at least one stratum disposed to transfer liquid;
providing at least one stratum disposed to absorb and store transferred liquid;
providing a liquid impermeable barrier stratum; and
causing at least one of the strata to contain an odor absorbent substance.

14. The method of claim 13, wherein the step of causing at least one of the strata to contain an odor absorbent substance comprises the further step of causing at least one of the strata to contain a weak base, wherein the weak base is selected from the group consisting of sodium bicarbonate and sodium carbonate.

15. A method of neutralizing acidic water in a water bearing bodily discharge, comprising the steps of:
providing at least one stratum disposed to transfer liquid;
providing at least one stratum disposed to absorb and store transferred liquid;
providing a liquid impermeable barrier stratum; and
causing at least one of the strata to contain an acid neutralizing substance.

16. The method of claim 15, wherein the step of causing at least one of the strata to contain an acid neutralizing substance comprises the further step of causing at least one of the strata to contain a weak base, wherein the weak base is selected from the group consisting of sodium bicarbonate and sodium carbonate.
